SEQUENCE LISTING (6 sequences)

SEQ ID NO: 1 - DNA, 473 bp, Homo sapiens
  source 1..473: mol_type = genomic DNA
cctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacaggtaggagccagctcttccct

SEQ ID NO: 2 - DNA, 18 bp, Homo sapiens
  source 1..18: mol_type = other DNA
cctccccctaaaatctca

SEQ ID NO: 3 - DNA, 22 bp, Homo sapiens
  source 1..22: mol_type = other DNA
agggaagagttggtttttattt

SEQ ID NO: 4 - DNA, 25 bp, Homo sapiens
  source 1..25: mol_type = other DNA
gaaggatgtttttttggatttattg

SEQ ID NO: 5 - DNA, 26 bp, Homo sapiens
  source 1..26: mol_type = other DNA
ctaatacttaaaacactctttctcat

SEQ ID NO: 6 - DNA, 27 bp, Homo sapiens
  source 1..27: mol_type = other DNA
taccaaaccaaaccaaccaaacctaaa

(19) United States
(12) Patent Application Publication
(10) Pub. No.: US 2026/0357330 A1
(43) Pub. Date: Nov. 12, 2026

(54) GATA 3 AS EPIGENETIC MARKER FOR THE IDENTIFICATION OF IMMUNE CELLS

(71) Applicant: PRECISION FOR MEDICINE GMBH, Berlin (DE)

(72) Inventors: Sven OLEK, Berlin (DE); Kati BOURQUAIN, Berlin (DE)

(21) Appl. No.: 19/487,543

(22) PCT Filed: Apr. 26, 2024
(86) PCT No.: PCT/EP2024/061517

§ 371 Date: Nov. 25, 2025

(30) Foreign Application Priority Data
May 26, 2023 (DE) 10 2023 114 004.3

(51) Int. Cl. C12Q 1/6844; C12Q 1/6813; C12Q 1/6869; C12Q 1/6881

(52) U.S. Cl. CPC C12Q 1/6844; C12Q 1/6813; C12Q 1/6869; C12Q 1/6881; C12Q 2600/106; C12Q 2600/154; G01N 2800/52

(57) ABSTRACT

Disclosed are methods, in particular an in vitro methods, for identifying immune cells in a sample, comprising analyzing the methylation status of at least one CpG position in the mammalian gene region for GATA Binding Protein 3 (GATA3) according to SEQ ID No. 1, wherein a demethylation or lack of methylation of said at least one CpG position in the gene region is indicative for T helper cells, cytotoxic T cells, Th2 cells, memory CD4+ T cells or memory CD8+ T cells, in particular Th2 cells. The methods can identify immune cells on an epigenetic level and distinguish them from all other cells in complex samples, such as, for example, other blood, non-blood or other immune cells. Disclosed are an improved methods for quantifying immune cells, in particular in complex samples. The methods can be performed without a step of purifying and/or enriching cells, preferably in whole blood and/or non-trypsinized tissue.

The present invention relates to a method, in particular an in vitro method, for identifying immune cells in a sample, comprising analyzing the methylation status of at least one CpG position in the mammalian gene region for GATA Binding Protein 3 (GATA3) according to SEQ ID No. 1, wherein a demethylation or lack of methylation of said at least one CpG position in the gene region is indicative for T helper cells, cytotoxic T cells, Th2 cells, memory CD4+ T cells or memory CD8+ T cells, in particular Th2 cells. The analysis according to the invention can identify immune cells on an epigenetic level and distinguish them from all other cells in complex samples, such as, for example, other blood, non-blood or other immune cells. The present invention furthermore provides an improved method for quantifying immune cells, in particular in complex samples. The method can be performed without a step of purifying and/or enriching cells, preferably in whole blood and/or non-trypsinized tissue.

Furthermore, the present invention relates to a kit for performing the above methods as well as respective uses thereof. It is one aim of this invention to provide a novel, more robust means to quantitatively detect and measure immune cells of the blood within any solid organs, tissue or body fluid of a mammal, in particular cord blood.

### BACKGROUND OF THE INVENTION

GATA binding protein 3, also known as GAT3, is a protein which belongs to the GATA family of transcription factors. The protein contains two GATA-type zinc fingers and is an important regulator of T-cell development and plays an important role in endothelial cell biology. Defects in this gene are the cause of hypoparathyroidism with sensorineural deafness and renal dysplasia.

Liu X L, et al. (in: TGFBR3 co-downregulated with GATA3 is associated with methylation of the GATA3 gene in bladder urothelial carcinoma. Anat Rec (Hoboken). 2013 November; 296 (11): 1717-23. doi: 10.1002/ar.22802. Epub 2013 Oct. 4. PMID: 24124001) performed quantitative real-time PCR to examine the mRNA expression of TGFBR3 and GATA3, and bisulfite genomic sequencing to evaluate the methylation status in TGFBR3 and GATA3 promoter regions in fresh tumor and the corresponding paracarcinoma tissues from 29 patients with bladder cancer (BUC). Their findings suggest that methylation in the GATA3 promoter region may inhibit the expression of GATA3 mRNA, which leads to the reduced expression of TGFBR3 mRNA in BUC.

Zhu Y, et al. (in: Aberrant DNA methylation of GATA binding protein 3 (GATA3), interleukin-4 (IL-4), and transforming growth factor-β (TGF-β) promoters in Behcet's disease. Oncotarget. 2017; 8 (38): 64263-64272. Published 2017 Jul. 22. doi: 10.18632/oncotarget. 19500) disclose that the promoter methylation level of GATA3, IL-4 and TGF-β was significantly up-regulated in active BD patients and negatively correlated with the corresponding mRNA expression. The mRNA expression of GATA3 and TGF-β was markedly down-regulated in active BD patients compared to healthy individuals. Treatment with corticosteroids and cyclosporine (CsA) resulted in a decrease of the methylation level of GATA3 and TGF-β in inactive BD patients.

WO 2017/075465A1 provides markers, marker signatures and molecular targets that correlate with dysfunction of immune cells and are advantageously independent of the immune cell activation status. The present markers, marker signatures and molecular targets provide for new ways to evaluate and modulate immune responses. Specifically, GATA3 and/or FOX01 modulation are provided for use as markers, marker signatures and molecular targets. Therapeutic methods are also provided to treat a patient in need thereof who would benefit from an increased immune response. Methylation is not mentioned.

US 2019-0117664A1 relates to methods and pharmaceutical composition for the treatment of T-helper type 2 (Th2)-mediated diseases. More particularly, the present invention relates to an inhibitor of the Suv39 h1-HP1a silencing pathway for use in the treatment of a T-helper type 2 (Th2)-mediated disease, in particular allergic asthma. GATA3 is mentioned as a Th2 “master regulator”. Methylation is not mentioned.

Even though almost all cells in an individual contain the exact same complement of DNA code, higher organisms must impose and maintain different patterns of gene expression in the various types of tissue. Most gene regulation is transitory, depending on the current state of the cell and changes in external stimuli. Persistent regulation, on the other hand, is a primary role of epigenetics-heritable regulatory patterns that do not alter the basic genetic coding of the DNA. DNA methylation is the archetypical form of epigenetic regulation; it serves as the stable memory for cells and performs a crucial role in maintaining the long-term identity of various cell types. Recently, other forms of epigenetic regulation were discovered. In addition to the “fifth base” 5-methylcytosine (mC), a sixth (5-hydroxymethylcytosine, hmC), seventh (5-formylcytosine, fC) and eighth (5-carboxycytosine, cC) can be found (Michael J. Booth et al. Quantitative Sequencing of 5-Methylcytosine and 5-Hydroxymethylcytosine at Single-Base Resolution Science 18 May 2012, Vol. 336 no. 6083 pp. 934-937).

The primary target of mentioned DNA modifications is the two-nucleotide sequence Cytosine-Guanine (a ‘CpG site’); within this context cytosine (C) can undergo a simple chemical modification to become formylated, methylated, hydroxymethylated, or carboxylated. In the human genome, the CG sequence is much rarer than expected, except in certain relatively dense clusters called ‘CpG islands’. CpG islands are frequently associated with gene promoters, and it has been estimated that more than half of the human genes have CpG islands (Antequera and Bird, Proc Natl Acad Sci USA 90:11995-9, 1993).

Aberrant methylation of DNA is frequently associated with the transformation from healthy to cancerous cells. Among the observed effects are genome-wide hypomethylation, increased methylation of tumor suppressor genes, and hypomethylation of many oncogenes (reviewed, for example, by Jones and Laird, Nature Genetics 21:163-167, 1999; Esteller, Oncogene 21:5427-5440, 2002; and Laird, Nature Reviews/Cancer 3:253-266, 2003). Methylation profiles have been recognized to be tumor specific (i.e., changes in the methylation pattern of particular genes or even individual CpGs are diagnostic of particular tumor types), and there is now an extensive collection of diagnostic markers for bladder, breast, colon, esophagus, stomach, liver, lung, and prostate cancers (summarized, for example, by Laird, Nature Reviews/Cancer 3:253-266, 2003).

For one of the recently described modification of cytosine, 5-hydroxymethylation, the utility of oxidative bisulfite sequencing to map and quantify 5hmC at CpG islands was shown (Michael J. Booth et al. Quantitative Sequencing of 5-Methylcytosine and 5-Hydroxymethylcytosine at Single-Base Resolution Science 18 May 2012, Vol. 336 no. 6083 pp. 934-937). High levels of 5hmC were found in CpG islands associated with transcriptional regulators and in long interspersed nuclear elements. It is suggested that these regions might undergo epigenetic reprogramming in embryonic stem cells.

WO 2012/162660 describes methods using DNA methylation arrays are provided for identifying a cell or mixture of cells and for quantification of alterations in distribution of cells in blood or in tissues, and for diagnosing, prognosing and treating disease conditions, particularly cancer. The methods use fresh and archival samples.

In view of the above, it is an object of the present invention to provide an improved and in particular specific and robust method based on DNA-methylation analysis as a superior tool in order to more conveniently and reliably detect, identify, discriminate, and quantify certain types of immune cells.

The present invention solves the above object by providing a method for identifying immune cells in a sample, comprising analyzing the methylation status of at least one CpG position in the mammalian gene region for GATA Binding Protein 3 (GATA3) according to SEQ ID No. 1, wherein a demethylation or lack of methylation of said at least one CpG position in the gene region to at least about 10% is indicative for T helper cells, cytotoxic T cells, Th2 cells, memory CD4+ T cells or memory CD8+ T cells, when compared to a naive CD8+ cell, activated cytotoxic T cell, activated T helper cell, naive CD4+ T cell, Th1 cell, granulocyte, B cell, NK-cell, or monocyte.

Preferred is the method according to the present invention, wherein a demethylation or lack of methylation of said at least one CpG position in the gene region to at least about 45% is indicative for memory CD8+ T cells or Th2 cells, when compared to a memory CD4+ T cell, memory CD8+ T cell, naive CD8+ T cell, activated cytotoxic T cell, activated T helper cell, naive CD4+ T cell, Th1 cell, granulocyte, B cell, NK-cell, or monocyte, or wherein a demethylation or lack of methylation of said at least one CpG position in the gene region to at least about 80% is indicative for Th2 cells.

The present invention is based on the surprising identification of a region of the GATA3 gene according to SEQ ID NO: 1 by the inventors as specific epigenetic marker, allowing the identification of immune cells as well as the clinical routine application of said analysis.

Therefore, the present invention further solves the above object by providing a method for identifying an immune cell population comprising CD8+ T cells, CD4+ T cells, memory CD4+ T cells, naive CD4+ T cells, Th1 cells and Th2 cells in a sample, comprising analyzing the methylation status of at least one CpG position in the mammalian gene region for GATA Binding Protein 3 (GATA3) according to SEQ ID No. 1, wherein said at least one CpG position is selected from CpG position 248, 268, 279, 293, 313, 316, 343, 350, 356, 368, 398, 410, 416, and 440 according to SEQ ID No. 1, wherein a demethylation or lack of methylation of said at least one CpG position in the gene region to at least about 10% is indicative for an immune cell of the population, i.e. CD8+ T cells, CD4+ T cells, memory CD4+ T cells, naive CD4+ T cells, Th1 cells and/or Th2 cells.

Preferred is the method according to the present invention for identifying an immune cell population comprising CD8+ T cells, CD4+ T cells, memory CD4+ T cells, and Th2 cells in a sample, comprising analyzing the methylation status of at least one CpG position in the mammalian gene region for GATA Binding Protein 3 (GATA3) according to SEQ ID No. 1, wherein said at least one CpG position is selected from CpG position 248, 268, 279, 293, 313, 316, 343, 350, 356, and 368 according to SEQ ID No. 1, wherein a demethylation or lack of methylation of said at least one CpG position in the gene region to at least about 20% is indicative for an immune cell of the population, i.e. CD8+ T cells, CD4+ T cells, memory CD4+ T cells, and/or Th2 cells.

More preferred is a method for identifying Th2 immune cells in a sample, comprising analyzing the methylation status of at least one CpG position in the mammalian gene region for GATA Binding Protein 3 (GATA3) according to SEQ ID No. 1, wherein said at least one CpG position is selected from CpG position 110, 118, 136, 145, 148, 155, 163, 172, 190, 194, 199, 204, and 212 according to SEQ ID No. 1, and preferably selected from CpG position 155, 163, 172, 190, 194, 199, 204, and 212 according to SEQ ID No. 1, wherein a demethylation or lack of methylation of said at least one CpG position in the gene region to at least about 80% is indicative for a Th2 immune cell.

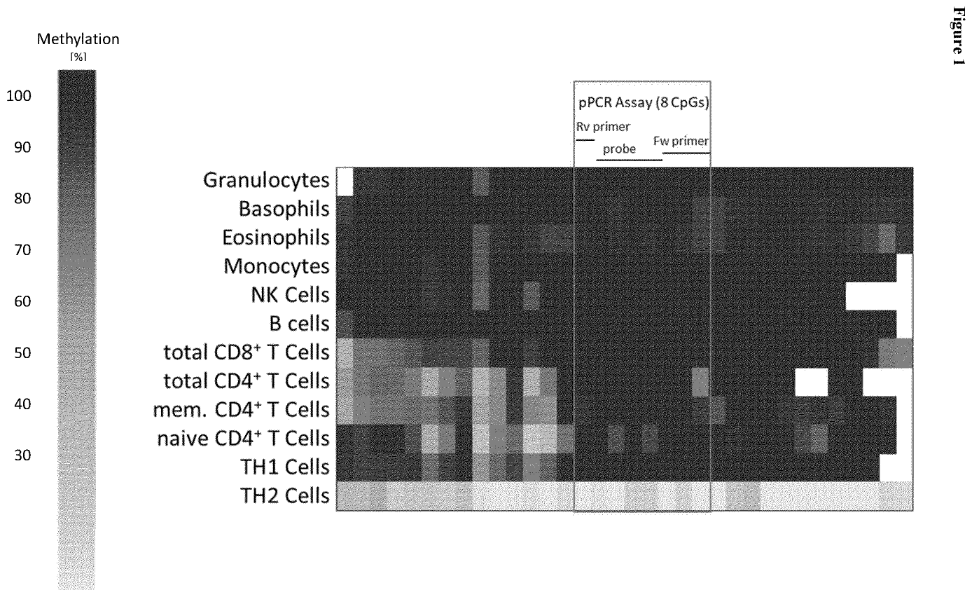

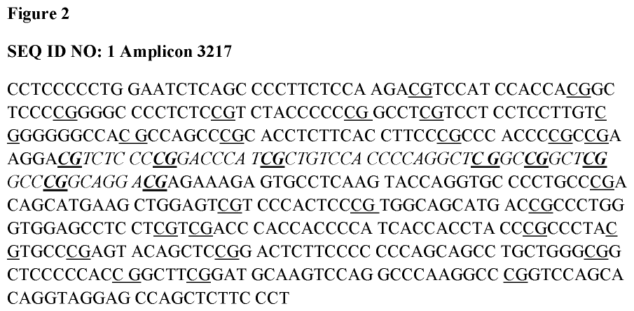

In the context of the invention the CpG positions are counted/numbered from the 5′-end of the sequence given in FIG. 2, below. CpGs are underlined. The positions as given correspond to the squares in the columns of FIG. 1, nevertheless, counted starting from the right side of the Figure (see also legend).

In the context of the present invention, the gene region shall comprise all of the genomic regions relating to and encoding for GATA3. Thus, included are enhancer regions, promoter region(s), introns, exons, and non-coding regions (5′- and/or 3′-regions) that belong to GATA3. Preferred is thus a method according to the present invention, wherein the at least one CpG position is present in the 5′ region upstream from the transcription start, promoter region, the 5′ or 3′ untranslated regions, exon, intron, exon/intron border and/or in the 3′ region downstream of the transcriptional stop of the gene as analyzed.

The sequence of Homo sapiens GATA-binding protein 3 (GATA3) can be taken from ENSG00000107485, the gene is located on chromosome 10:8,045,378-8,075,198 forward strand.

In the context of the present invention, the genomic region for Homo sapiens GATA Binding Protein 3 (GATA3), in particular according to SEQ ID No. 1 (Amp 3217), allows for the identification of T helper cells, cytotoxic T cells, Th2 cells, memory CD4+ T cells and/or memory CD8+ T cells, when compared to a naive CD8+ cell, activated cytotoxic T cell, activated T helper cell, naive CD4+ T cell, Th1 cell, granulocyte, B cell, NK-cell, or monocyte, and preferably memory CD8+ T cells and/or Th2 cells, when compared to a memory CD4+ T cell, memory CD8+ T cell, naive CD8+ T cell, activated cytotoxic T cell, activated T helper cell, naive CD4+ T cell, Th1 cell, granulocyte, B cell, NK-cell, or monocyte, or more preferably the identification of Th2 cells. Surprisingly, the discriminatory pattern of bisulfite convertible and non-convertible cytosine is particularly and even exclusively limited to the genomic region of GATA3, in particular the amplicon according to SEQ ID No. 1.

The inventors could demonstrate that in TH2 cells the CpG motifs as disclosed are almost completely demethylated (i.e. to more than about 80%) and demethylated in CD8+ T cells or Th2 cells (i.e. to more than about 45%), and demethylated in T helper cells, cytotoxic T cells, Th2 cells, memory CD4+ T cells or memory CD8+ T cells (i.e. to more than about 10%), when compared to other immune cells, such as naive CD8+ T cells, activated cytotoxic T cells, activated T helper cells, naive CD4+ T cells, Th1 cells, granulocytes, B cells, NK-cells, and monocytes, where the same motifs are nearly completely, and preferably completely, methylated.

The inventors could further demonstrate that in TH2 cells the CpG motifs selected from CpG position 110, 118, 136, 145, 148, 155, 163, 172, 190, 194, 199, 204, and 212 according to SEQ ID No. 1, and preferably selected from CpG position 155, 163, 172, 190, 194, 199, 204, and 212 according to SEQ ID No. 1, are almost completely demethylated, i.e. to at least about 80%. The region distinctive for Th2 is preferred, includes positions 155, 163, 172, 190, 194, 199, 204, and 212 according to SEQ ID No. 1, and forms the basis of a preferred qPCR assay, see examples and FIG. 1, comprising the oligomers according to SEQ ID Nos. 4 to 6.

Furthermore, in CD8+ T cells, CD4+ T cells, memory CD4+ T cells, naive CD4+ T cells, Th1 cells and Th2 cells, the CpG motifs selected from CpG position 248, 268, 279, 293, 313, 316, 343, 350, 356, 368, 398, 410, 416, and 440 according to SEQ ID No. 1 are demethylated to at least about 10%, thus indicating cells of this population. Similarly, in CD8+ T cells, CD4+ T cells, memory CD4+ T cells, and Th2 cells, the CpG motifs selected from CpG position 248, 268, 279, 293, 313, 316, 343, 350, 356, and 368 according to SEQ ID No. 1, are demethylated to at least about 20%, thus indicating cells of this population, when compared to granulocytes, basophils, eosinophils, monocytes, NK cells, B cells, naive CD4+ T cells, and Th1 cells.

The differential methylation of the CpG motifs within the aforementioned regions is a valuable tool to identify the immune cell populations as indicated, such as will be required/or at least of some value for identifying and quantifying said cells in autoimmune diseases, transplant rejections, infection diseases, cancer, allergy, endometriosis, cardiovascular diseases, primary and secondary immunodeficiencies, such as, for example, HIV infections and AIDS, Graft versus Host (GvH), hematologic malignancies, rheumatoid arthritis, multiple sclerosis, or a cytotoxic T cell related immune status in any envisionable diagnostic context. The assay allows measurement of immune cells without purification or any staining procedures.

Another preferred aspect of the method according to the present invention then further comprises a quantification of the relative amount of Th2 cells based on comparing relative amounts of said methylation frequency in the genetic region for GATA3, preferably according to SEQ ID No. 1, more preferably selected from CpG position 110, 118, 136, 145, 148, 155, 163, 172, 190, 194, 199, 204, and 212 according to SEQ ID No. 1, and most preferably selected from CpG position 155, 163, 172, 190, 194, 199, 204, and 212 according to SEQ ID No. 1, as analyzed with relative amounts of the methylation frequency in a control gene, such as, for example, GAPDH. Said quantification is thus achieved based on the ratio of the bisulfite convertible DNA to non-convertible DNA in the genetic regions for GATA3 (e.g. of SEQ ID No. 1) as described and analyzed herein. Most preferred is a quantification of the relative amount of immune cells is based on an (preferably parallel or simultaneous) analysis of the relative amount of bisulfite convertible DNA of the cell-specific regions for GATA3 (in particular according to SEQ ID NO: 1), and of the relative amount of bisulfite convertible DNA of cell-unspecific genes (preferably designated “control genes” or “control regions”, such as, for example, the gene for GAPDH). Preferably, quantitative PCR (qPCR) is used for the quantification.

In a further preferred embodiment of the method according to the present invention, said analysis of bisulfite convertibility comprises amplification with at least one primer of suitable primer pairs that can be suitably designed based on SEQ ID No. 1, preferably oligomers according to any of SEQ ID No. 2 to 6.

In contrast to flow cytometry and mRNA measurements, using the methods according to the present invention, the measurement(s) and analyses can be done independent of purification, storage—and to quite some extent—also to tissue quality.

Preferably, the amplification involves a polymerase enzyme, a PCR or chemical amplification reaction, or other amplification methods as known to the person of skill as described below, e.g. in the context of MSP, HeavyMethyl, Scorpion, MS-SNUPE, MethylLight, bisulfite sequencing, methyl specific restriction assays and/or digital PCR (see, for example Kristensen and Hansen PCR-Based Methods for Detecting Single-Locus DNA Methylation Biomarkers in Cancer Diagnostics, Prognostics, and Response to Treatment Clinical Chemistry 55:8 1471-1483 (2009)).

With the amplification, an amplicon of the GATA3 gene region is produced that is a particularly preferred “tool” for performing the method(s) according to the present invention. Consequently, oligomers according to any of SEQ ID No. 2 to 6 or an amplicon as amplified by a primer pair based on SEQ ID No. 2 and 3 or 4 and 5 as mentioned herein constitute preferred embodiments of the present invention. Thus, the sequence of SEQ ID No. 1 (and, if needed, the complementary sequences thereto) can be used to design primers for amplifications, i.e. serve as “beacons” in the sequence as relevant. Similarly, additional primers and probes can be designed based on the amplicon according to SEQ ID No. 1. Amplification can take place either in the genomic and/or bisulfite (i.e. “converted”) DNA sequence.

The present invention furthermore relates to a method for producing an (isolated) amplicon sequence in the mammalian (e.g. human) gene region for GATA Binding Protein 3 (GATA3), preferably according to SEQ ID No. 1 of T helper cells, cytotoxic T cells, Th2 cells, memory CD4+ T cells or memory CD8+ T cells, comprising a bisulfite treatment of the genomic DNA derived from a mammalian (e.g. human) sample comprising immune cells, and amplification of said gene region or a part thereof (e.g. located according to the sequence of SEQ ID No. 1), wherein said amplicon sequence exhibits a demethylation or lack of methylation of at least one CpG position in the amplicon to at least about 10%, when compared to a naive CD8+ T cell, activated cytotoxic T cell, activated T helper cell, naive CD4+ T cell, Th1 cell, granulocyte, B cell, NK-cell, or monocyte. The amplicon sequence exhibits a demethylation or lack of methylation of at least one CpG position in the amplicon to at least about 45% in memory CD8+ T cells and/or Th2 cells, when compared to a memory CD4+ T cell, memory CD8+ T cell, naive CD8+ T cell, activated cytotoxic T cell, activated T helper cell, naive CD4+ T cell, Th1 cell, granulocyte, B cell, NK-cell, or monocyte. The amplicon sequence exhibits a demethylation or lack of methylation of at least one CpG position in the amplicon to at least about 80%, preferably at least about 90%, in Th2 cells, when compared to immune cells other than Th2 cells. Again, preferably quantitative PCR (qPCR) can be used for the quantification, either of one CpG position or the overall demethylation of the region as analyzed.

The present invention furthermore relates to a method for producing an (isolated) amplicon sequence in the mammalian (e.g. human) gene region for GATA Binding Protein 3 (GATA3), preferably according to SEQ ID No. 1 of CD8+ T cells, CD4+ T cells, memory CD4+ T cells, naive CD4+ T cells, Th1 cells and Th2 cells, comprising a bisulfite treatment of the genomic DNA derived from a mammalian (e.g. human) sample comprising immune cells, and amplification of said gene region or a part thereof (e.g. located according to the sequence of SEQ ID No. 1), wherein said amplicon sequence exhibits a demethylation or lack of methylation of at least one CpG position selected from CpG position 248, 268, 279, 293, 313, 316, 343, 350, 356, 368, 398, 410, 416, and 440 according to SEQ ID No. 1 to at least about 10%, when compared to a naive CD8+ T cell, activated cytotoxic T cell, activated T helper cell, naive CD4+ T cell, Th1 cell, granulocyte, B cell, NK-cell, or monocyte. The amplicon sequence exhibits a demethylation or lack of methylation of at least one CpG position in the amplicon to at least about 20% in CD8+ T cells, CD4+ T cells, memory CD4+ T cells, and Th2 cells at least one CpG position is selected from CpG position 248, 268, 279, 293, 313, 316, 343, 350, 356, and 368 according to SEQ ID No. 1, when compared to a granulocyte, basophil, eosinophil, monocyte, NK cell, B cell, naive CD4+ T cell, and Th1 cell.

The present invention furthermore relates to a method for producing an (isolated) amplicon sequence in the mammalian (e.g. human) gene region for GATA Binding Protein 3 (GATA3), preferably according to SEQ ID No. 1 of Th2 cells, wherein said amplicon sequence exhibits a demethylation or lack of methylation of at least one CpG position selected from CpG position 110, 118, 136, 145, 148, 155, 163, 172, 190, 194, 199, 204, and 212 according to SEQ ID No. 1, and preferably selected from CpG position 155, 163, 172, 190, 194, 199, 204, and 212 according to SEQ ID No. 1 to at least about 80%, preferably at least about 90%, when compared to another immune cell.

The person of skill will furthermore be able to select specific subsets of CpG positions in order to minimize the amount of sites to be analyzed, for example at least one of CpG position selected from a CpG position in an amplicon according to SEQ ID No. 1, and is preferably selected from the CpG positions 248, 268, 279, 293, 313, 316, 343, 350, 356, 368, 398, 410, 416, and 440 in the amplicon AMP 3217 according to SEQ ID No. 1, and is more preferably selected from CpG positions 248, 268, 279, 293, 313, 316, 343, 350, 356, and 368 in the amplicon AMP 3217 according to SEQ ID No. 1, and is most preferably selected from CpG positions 155, 163, 172, 190, 194, 199, 204, and 212 according to SEQ ID No. 1, optionally with at least one of positions 110, 118, 136, 145, and 148.

Preferred are combinations of CpG positions 155, 163, 172, 190, 194, 199, 204, and 212 for distinguishing Th2 cells from other blood (immune) cells. Preferred are combinations of 3, 4, 5, 6, 7, or 8 positions, the analysis of which produces sufficient data and/or information in order to be informative in the context of the present invention.

In order to analyze the bisulfite convertibility of CpG positions, any known method to analyze DNA methylation can be used. In a preferred embodiment of the method according to the present invention, the analysis of the methylation status comprises a method selected from methylation specific enzymatic digests, bisulphite sequencing, analysis selected from promoter methylation, CpG island methylation, MSP (methylation-specific PCR), HeavyMethyl, MethyLight, Ms-SNuPE or other methods relying on a detection of amplified DNA. These methods are well known to the person of skill, and can be found in the respective literature.

In a preferred embodiment of the method according to the present invention, said method is suitable for routine application, for example on a DNA-such as an oligonucleotide-based chip. Based on the above information and the respective literature, the person of skill will be able to adjust the method as above to such settings.

In yet another preferred embodiment of the methods according to the present invention, said method is performed without a step of purifying and/or enriching said cells to be identified, preferably using whole blood and/or non-trypsinized tissue.

In another preferred embodiment of the method according to the present invention, the identification comprises a distinction of said immune cells as above from all major peripheral blood cell types and/or non-blood cells, or cord blood cells, and/or non-blood cells such as from at least one of the cell type selected from endothelial cells, smooth muscle cells (aortic or intestine), and dermal fibroblasts.

In yet another preferred embodiment of the method according to the present invention, the sample is selected from a mammalian body fluid, including human blood samples, human samples comprising immune cells, cord blood sample, or a tissue, organ or a sample of lymphocytes or a purified or separated fraction of such tissue, organ or lymphocytes or a cell type sample. Preferably, said mammal is a mouse, goat, dog, pig, cat, cow rat, monkey or human. The samples can be suitably pooled, if required. Preferably, said cells are human cells, such as immune cell preparations.

Another preferred aspect of the method according to the present invention then further comprises the step of concluding on the immune and/or disease status of said mammal based on said immune cells as identified. The immune cells as identified can be quantified and be used as a benchmark to relatively quantify further detailed subpopulations, or it can be used as a predictive and/or screening and/or diagnostic and/or prognostic and/or adverse events detecting factor, or it can be used to finally detect this population to determine the overall immune or disease activity status.

In yet another preferred embodiment of the methods according to the present invention, the mammal suffers from or is likely to suffer from autoimmune diseases, transplant rejections, infection diseases, cancer, and/or allergy as but not limited to Trypanosoma cruzi-infection, malaria and HIV infection; hematologic malignancies, for example, but not limited to chronic myelogenous leukemia, multiple myeloma, non Hodgkin's lymphoma, Hodgkin's Disease, chronic lymphocytic leukemia, Graft versus Host and Host versus Graft Disease, mycosis fungoides, extranodal T cell lymphoma, cutaneous T cell lymphomas, anaplastic large cell lymphoma, angioimmunoblastic T cell lymphoma and other T-cell, B-cell and NK cell neoplasms, endometriosis, cardiovascular diseases, T cell deficiencies such as but not limited to lymphocytopenia, severe combined immunodeficiency (SCID), Omenn syndrome, cartilage-hair hypoplasia, acquired immune deficiency syndrome (AIDS), and hereditary conditions such as DiGeorge syndrome (DGS), chromosomal breakage syndromes (CBSs), multiple sclerosis, rheumatoid arthritis, systemic lupus erythematosus, Sjögren's syndrome, systemic sclerosis, dermatomyositis, primary biliary cirrhosis, primary sclerosing cholangitis, ulcerative colitis, Crohn's disease, psoriasis, vitiligo, bullous pemphigoid, alopecia areata, idiopathic dilated cardiomyopathy, type 1 diabetes mellitus, Graves' disease, Hashimoto's thyroiditis, myasthenia gravis, IgA nephropathy, membranous nephropathy, and pernicious anemia; and B-cell and T-cell combined disorders such as but not limited to ataxia telangiectasia (AT) and Wiskott-Aldrich syndrome (WAS); and carcinomas such as but not limited to breast cancer, colorectal cancer, gastric cancer, pancreatic cancer, hepatocellular carcinoma, cholangiocarcinoma, melanoma, and head and neck cancer.

Another preferred aspect of the method according to the present invention then relates to a method as above, further comprising measuring and/or monitoring the amount of the immune cells as identified in response to chemical and/or biological substances that are provided to said mammal, i.e. in response to a treatment of said patient. Said method comprises the steps as above, and comparing said relative amount of said cells as identified to a sample taken earlier or in parallel from the same mammal, and/or to a control sample. Based on the results as provided by the method(s) of the invention, the attending physician will be able to conclude on the immune status of the patient, and adjust a treatment of the underlying disease accordingly.

Preferably, said method is performed without a step of purifying and/or enriching cells, preferably in whole blood and/or non-trypsinized tissue, or any other biological sample potentially containing said immune cells as e.g. a sample for cell transfer into a patient.

Another preferred aspect of the method according to the present invention then relates to a method as above, further comprising formulating said immune cells as identified for transplantation into a patient. Pharmaceutical preparations for these purposes and methods for their production are performed according to methods known in the art of transplantation medicine.

Another preferred aspect of the method according to the present invention then further then relates to a method for treating a condition or disease in a mammal, in particular in a human, comprising a method according to the invention as above, and the step of transplanting the immune cells as identified, i.e. T helper cells, cytotoxic T cells, Th2 cells, memory CD4+ T cells or memory CD8+ T cells, preferably Th2 cells, and/or as isolated/multiplied in cell culture into a patient. Pharmaceutical preparations for these purposes and methods for their production are performed according to methods known in the art of transplantation medicine. The transplant can be autologous or allogenic.

Another preferred aspect of the method according to the present invention then further then relates to a method for treating and/or preventing a condition or disease in a mammal, in particular in a human, comprising a method according to the invention as above including a suitable treatment for said condition or disease comprising providing chemical and/or biological substances as above, and adjusting said treatment of the underlying disease or condition based on the results as provided by the method(s) of the invention. This may comprise the step of concluding on the immune or disease status of said mammal based on said immune cells as identified. The immune cells can be quantified and be used as a benchmark to relatively quantify further detailed subpopulations, or it can be used as a predictive and/or screening and/or diagnostic and/or prognostic and/or adverse events detecting factor, or it can be used to finally detect this population to determine the overall immune activity status. This basis allows for adjusting said treatment, if necessary. Such adjustments may comprise the step of transplanting the immune cells as identified and isolated/multiplied in cell culture into a patient as above, and/or providing additional chemical and/or biological substances for adjusting said treatment and/or prevention.

One particular example is a method for treating and/or preventing a condition or disease in a mammal, in particular in a human, wherein first a medicament is provided to said mammal. Respective medication strategies are known, optionally with suitable carriers and adjuvants. Therefore, the method then comprises measuring and/or monitoring the amount of immune cells in response to said medication that is/are provided to said mammal. In case of an insufficient number of immune cells, said treatment (here: medication) is adjusted, i.e. more drug is given. The method may be repeated until sufficient desired cells (i.e. a substantial population of immune cells) can be detected.

Treatment and/or prevention shall herein relate to the curing, prevention or alleviation of a disorder or malfunction of the body, i.e. bringing a body back to its healthy state.

Pharmaceutical preparations for these purposes and methods for their production are performed according to methods known in the art of a treatment using chemical and/or biological substances or transplantation medicine. Again, the transplant can be autologous or allogenic.

Another preferred aspect of the method according to the present invention relates to an oligomer according to any of SEQ ID No. 2 to 6, or an amplicon according to SEQ ID No. 1.

Yet another preferred aspect of the present invention then relates to a kit for identifying, quantifying, and/or monitoring immune cells, in particular T helper cells, cytotoxic T cells, Th2 cells, memory CD4+ T cells or memory CD8+ T cells, and preferably Th2 cells, in a mammal based on the analysis of the bisulfite accessibility of CpG positions in the genetic region for GATA3, in particular according to SEQ ID NO: 1, comprising components for performing a method according to invention as described herein, in particular a kit comprising a) a bisulfite reagent, and b) materials for the analysis of the methylation status of CpG positions selected from the CpG positions in the region according to SEQ ID NO: 1, such as an oligomer selected from the sequences according to SEQ ID No. 2 to 6, or an amplicon as amplified by a primer pair based on SEQ ID No. 2 and 3 or 4 and 5, respectively.

The present invention also encompasses the use of oligomers or amplicon or a kit according to the present invention for identifying and/or for monitoring immune cells in a mammal as described herein, in particular Th2 cells.

As mentioned above, recently three new cytosine modifications were discovered. Therefore, it is expected that future scientific findings will correct epigenetic patterns of modification described in the past. These past patterns of cytosine modification encompass bisulfite convertible (non-methylated, non-modified) and non-convertible (methylated, modified) cytosine. Both termini need to be corrected, as described. According to the novel scientific findings (i) non-bisulfite convertible cytosine encompasses 5-methylcytosine (mC) and 5-hydroxymethylcytosine (hmC), and (ii) bisulfite convertible (i.e. the “bisulfite convertibility”) cytosine encompasses 5-formylcytosine (fC), 5-carboxycytosine (cC), as well as non-modified cytosine.

Additionally, past inventions are based on (i) the ratio of bisulfite convertible cytosine to whole amount of chromatin (cell-type independent, 100% bisulfite convertible DNA locus) or (ii) on the ratio of bisulfite convertible cytosine (fC, cC, non-modified cytosine) to non-bisulfite convertible cytosine (hmC and mC). These ratios characterize cell type, cell differentiation, cell stage as well as pathological cell stages. Therefore, new techniques will result in novel, more specific ratios and might supplement current cell specific, cell state specific as well as pathological patterns of epigenetic modifications and therefore, define potential novel biomarkers. Novel ratios to be discovered as biomarkers can be defined as:

Biomarker Ratio=a/b
a=Σ(C and/or mC and/or hmC and/or fC and/or cC)
b=Σ(C and/or mC and/or hmC and/or fC and/or cC),
whereby a and b differ from each other by one to four kinds of modifications. Discovery of novel DNA modifications will enlarge this enumeration.

For the purpose of definition for the present application, “epigenetic modifications” in the DNA sequence is referred to by the terminology of (i) bisulfite convertible cytosine (5-formylcytosine, (fC) and/or 5-carboxycytosine (cC)) and (ii) non-bisulfite convertible cytosine ((including 5-methylcytosine (mC), 5-hydroxymethylcytosine, (hmC)). As both kinds of methylation, mC and hmC, are not bisulfite convertible, it is not possible to distinguish between these two. Likewise, fC, cC as well as non-modified cytosine are bisulfite convertible and can also not be distinguished from each other as well. The term “methylated” DNA encompasses mC as well as hmC. The term “non-methylated” DNA encompasses fC, cC, and non-modified DNA. It is expected that novel variants of DNA modifications will be discovered in future. Each type of modification will be either bisulfite convertible or not. However, since the present method reliably distinguishes between the two groups, these novel modifications will also be usable as markers.

Furthermore, apart from the modifications of DNA, also histones undergo posttranslational modifications that alter their interaction with DNA and nuclear proteins. Modifications include methylation, acetylation, phosphorylation, ubiquitination, sumoylation, citrullination, and ADP-ribosylation. The core of the histones H2A, H2B, and H3 can also be modified. Histone modifications act in diverse biological processes such as gene regulation, DNA repair, chromosome condensation (mitosis) and spermatogenesis (meiosis). Also for these modifications a specific pattern of modification is specific for different cell types, cell stages, differentiation status and such a pattern can be analyzed for bisulfite convertibility or similar methods in order to identify certain cells and cell stages. The present invention also encompasses a use of these modifications.

In summary, using the GATA3 genetic region and in particular the amplicon according to SEQ ID NO: 1 as described herein as a marker, more particularly the region of the qPCR assay comprising the 8 CpGs as described herein, the inventors very specifically identified, quantified and in particular differentiated immune cells, and in their relation to other cell types in a sample, for example to other blood cells.

The invention will now be further described in the following examples and with reference to the accompanying figures and the sequence listing, without being limited thereto. For the purposes of the present invention, all references as cited herein are incorporated by reference in their entireties.

FIG. 1 shows the analysis of CpG sites on amplicon AMP3217 (SEQ ID No. 1) according to the invention. The rows in the table correspond to the cell types as analyzed and the columns correspond to the CpG positions in the amplicon as analyzed (e.g. CpG 1, 2, etc.) with the positions indicated (AMP3217:110 corresponding to CpG at position 110 of Amplicon 3217 according to SEQ ID No. 1, . . . etc.), starting from the right side of the figure.

FIG. 2 shows the genomic sequence of the amplicon (AMP3217) according to the present invention (SEQ ID No. 1) with CpG positions underlined. The 8 positions of the assay according to the invention (positions 155, 163, 172, 190, 194, 199, 204, 212) are indicated in bold.

SEQ ID No. 1 shows the genomic sequence of amplicon No. 3217.

SEQ ID Nos. 2 to 6 show the sequences of specific oligomers (primers and probes) according to the present invention.

### EXAMPLES

### Example 1

In order to identify immune cells, qPCR was performed on bisulphite converted samples stemming from the human genomic region according to the sequence SEQ ID No. 1 (see FIGS. 1 and 2). For the actual epigenetic profiling of the amplicon region, the immune cell populations as analyzed were as shown in FIG. 1.

The bisulfite-converted target-regions of preferred qPCR-assay-system as developed were: Oligonucleotides for Bisulfite Sequencing (5′-3′)

Forward Primer-
(SEQ ID NO: 2)
CCTCCCCCTAAAATCTCA
Reverse Primer-
(SEQ ID NO: 3)
AGGGAAGAGTTGGTTTTTATTT

Oligonucleotides of qPCR Assay (TpG Variant i.e., demethylation-specific; 5′-3′)

Forward Primer-
(SEQ ID NO: 4)
GAAGGATGTTTTTTTGGATTTATTG
Reverse Primer-
(SEQ ID NO: 5)
CTAATACTTAAAACACTCTTTCTCAT
Probe-
(SEQ ID NO: 6)
TACCAAACCAAACCAACCAAACCTAAA

The analysis of the demethylation status of purified (immune) cell preparations with the GATA3 qPCR assay was found to be as follows (Table 1). Cell types to be identified according to the invention are indicated in bold:

| | | TpG | GAPDH | % GATA3 | % GATA3 |
|---|---|---|---|---|---|
| Description | Sorting | copies | copies | calibrated | Conservative |
| T helper | CD3+ CD4+ CD8− | 161 | 1900 | 9.8 | 16.0 |
| cells | | | | | |
| T helper | CD3+ CD4+ CD8− | 103 | 1690 | 7.0 | 11.4 |
| cells | | | | | |
| T helper | CD3+ CD4+ CD8− | 77 | 1783 | 5.0 | 9.0 |
| cells | | | | | |
| T helper | CD3+ CD4+ CD8− | 113.7 | 1791 | 7.3 | 12.1 |
| cells | | | | | |
| (average) | | | | | |
| Cytotoxic T | CD3+ CD4− CD8+ | 260 | 1433 | 21.1 | 27 |
| cells | | | | | |
| Cytotoxic T | CD3+ CD4− CD8+ | 62 | 1747 | 4.1 | 5.2 |
| cells | | | | | |
| Cytotoxic T | CD3+ CD4− CD8+ | 49 | 2167 | 2.6 | 3.5 |
| cells | | | | | |
| Cytotoxic T | CD3+ CD4− CD8+ | 123.7 | 1782.3 | 9.3 | 11.9 |
| cells | | | | | |
| (average) | | | | | |
| Monocytes | CD14+ | ND | 1733 | 0 | 0 |
| NK cells | CD3−/CD56+/CD16+ | 4 | 1710 | 0.3 | 0.4 |
| NK cells | CD3−/CD56+/CD16+ | 4 | 1370 | 0.3 | 0.4 |
| B cells | CD19+ | ND | 321 | 0 | 0 |
| Granulocytes | CD15++ | ND | 1343 | 0 | 0 |
| Eosinophil | CD15++/CD16− | 6 | 1923 | 0.4 | 0.5 |
| Granulocytes | | | | | |
| Basophil | CD3−/CD19−/CD14−/ | 3 | 1460 | 0.3 | 0.4 |
| Granulocytes | HLA-DR−/ | | | | |
| | CD123++/CD203c+ | | | | |
| Th1 cells | CD3+/CD4+/CD45RA−/ | 38 | 1610 | 2.8 | 4.5 |
| | CCR6−/CXCR3+ | | | | |
| Th1 cells | CD3+/CD4+/CD45RA−/ | 62 | 1397 | 5.2 | 10.8 |
| | CCR6−/CXCR3+ | | | | |
| Th2 cells | CD4+/CRTH2+ | 409 | 444 | 106.8 | 93.1 |
| Th2 cells | CD3+/CD4+/CD45RA−/ | 694 | 1327 | 60.7 | 75.7 |
| | CCR6−/CXCR3− | | | | |
| Th2 cells | CD3+/CD4+/CD45RA−/ | 551.5 | 885.5 | 83.75 | 84.4 |
| (average) | CCR6−/CXCR3− | | | | |
| Naïve CD4+ | CD4+/CD45RA+/CCR7− | 6 | 1817 | 0.4 | 0.7 |
| T cells | | | | | |
| Naïve CD4+ | CD4+/CD45RA+/CCR7− | 10 | 1517 | 0.8 | 1.4 |
| T cells | | | | | |
| Naïve CD4+ | CD4+/CD45RA+/CCR7− | 6 | 1650 | 0.4 | 0.7 |
| T cells | | | | | |
| Memory | CD4+/CD45RA+/CCR7+ | 192 | 1437 | 15.8 | 23.3 |
| CD4+ T | | | | | |
| cells | | | | | |
| Memory | CD4+/CD45RA+/CCR7+ | 94 | 1270 | 8.7 | 14.3 |
| CD4+ T | | | | | |
| cells | | | | | |
| Memory | CD4+/CD45RA+/CCR7+ | 70 | 1583 | 5.2 | 8.9 |
| CD4+ T | | | | | |
| cells | | | | | |
| Memory | CD4+/CD45RA+/CCR7+ | 118.7 | 1430 | 9.9 | 15.5 |
| CD4+ T | | | | | |
| cells | | | | | |
| (average) | | | | | |
| Activated T | CD4+ CD69+ PD1− | 17 | 1693 | 1.2 | 2.1 |
| helper cells | (TFH−) | | | | |
| Activated | CD8+ CD69+ PD1− | 21 | 1910 | 1.3 | 1.7 |
| cytotoxic T | (TFH−) | | | | |
| cells | | | | | |
| Naïve CD8+ | CD3+/CD4−/ | 1 | 1577 | 0.05 | 0.06 |
| T cells | CD8+/CD45RA+/CCR7+ | | | | |
| Naïve CD8+ | CD3+/CD4−/ | 0 | 2027 | 0 | 0.01 |
| T cells | CD8+/CD45RA+/CCR7+ | | | | |
| Memory | CD3+/CD4−/ | 336 | 2017 | 17.9 | 26.4 |
| CD8+ T | CD8+/CD45RA−/CCR7+ | | | | |
| cells | | | | | |
| Memory | CD3+/CD4−/ | 1003 | 1933 | 55.5 | 68.4 |
| CD8+ T | CD8+/CD45RA−/CCR7+ | | | | |
| cells | | | | | |
| Memory | CD3+/CD4−/ | 669.5 | 1975 | 36.7 | 47.4 |
| CD8+ T | CD8+/CD45RA−/CCR7+ | | | | |
| cells | | | | | |
| (memory) | | | | | |

1. A method for identifying immune cells in a sample, comprising analyzing the methylation status of at least one CpG position in the mammalian gene region for GATA Binding Protein 3 (GATA3) according to SEQ ID No. 1, wherein a demethylation or lack of methylation of said at least one CpG position in the gene region to at least about 10% is indicative for T helper cells, cytotoxic T cells, Th2 cells, memory CD4+ T cells or memory CD8+ T cells, when compared to a naive CD8+ T cell, activated cytotoxic T cell, activated T helper cell, naive CD4+ T cell, Th1 cell, granulocyte, B cell, NK-cell, or monocyte.

2. The method according to claim 1, wherein a demethylation or lack of methylation of said at least one CpG position in the gene region to at least about 45% is indicative for memory CD8+ T cells or Th2 cells, when compared to a memory CD4+ T cell, memory CD8+ T cell, naive CD8+ T cell, activated cytotoxic T cell, activated T helper cell, naive CD4+ T cell, Th1 cell, granulocyte, B cell, NK-cell, or monocyte, or wherein a demethylation or lack of methylation of said at least one CpG position in the gene region to at least about 80% is indicative for Th2 cells.

3. A method for identifying an immune cell population comprising CD8+ T cells, CD4+ T cells, memory CD4+ T cells, naive CD4+ T cells, Th1 cells and Th2 cells in a sample, comprising analyzing the methylation status of at least one CpG position in the mammalian gene region for GATA Binding Protein 3 (GATA3) according to SEQ ID No. 1, wherein said at least one CpG position is selected from CpG position 248, 268, 279, 293, 313, 316, 343, 350, 356, 368, 398, 410, 416, and 440 according to SEQ ID No. 1, wherein a demethylation or lack of methylation of said at least one CpG position in the gene region to at least about 10% is indicative for an immune cell of the population.

4. A method for identifying an immune cell population comprising CD8+ T cells, CD4+ T cells, memory CD4+ T cells, and Th2 cells in a sample, comprising analyzing the methylation status of at least one CpG position in the mammalian gene region for GATA Binding Protein 3 (GATA3) according to SEQ ID No. 1, wherein said at least one CpG position is selected from CpG position 248, 268, 279, 293, 313, 316, 343, 350, 356, and 368 according to SEQ ID No. 1, wherein a demethylation or lack of methylation of said at least one CpG position in the gene region to at least about 20% is indicative for an immune cell of the population.

5. A method for identifying Th2 immune cells in a sample, comprising analyzing the methylation status of at least one CpG position in the mammalian gene region for GATA Binding Protein 3 (GATA3) according to SEQ ID No. 1, wherein said at least one CpG position is selected from CpG position 110, 118, 136, 145, 148, 155, 163, 172, 190, 194, 199, 204, and 212 according to SEQ ID No. 1, and preferably selected from CpG position 155, 163, 172, 190, 194, 199, 204, and 212 according to SEQ ID No. 1, wherein a demethylation or lack of methylation of said at least one CpG position in the gene region to at least about 80% is indicative for a Th2 immune cell.

6. The method according to claim 1, wherein
said at least one CpG position is present in the 5′ region upstream from the transcription start, promoter region, the 5′ or 3′ untranslated regions, exon, intron, exon/intron border and/or in the 3′ region downstream of the transcriptional stop of said gene region as analyzed.

7. The method according to claim 1, wherein
said analysis of the bisulfite convertibility comprises a method selected from a methylation specific enzymatic digest, bisulfite sequencing, an analysis selected from promoter methylation, CpG island methylation, MSP, HeavyMethyl, MethyLight, Ms-SNuPE, and other methods relying on a detection of amplified DNA.

8. The method according to claim 5, further comprising a quantification of the relative amount of Th2 cells based on comparing relative amounts of said methylation frequency in the genetic region for GATA3 according to SEQ ID No. 1 as analyzed with relative amounts of the methylation frequency in a control gene, such as, for example, GAPDH.

9. The method according to claim 1, wherein
said sample is selected from a mammalian body fluid, including human blood samples, in particular a cord blood sample, or a tissue, organ or cell type blood sample, a sample of cord blood or a fraction thereof, a sample comprising immune cells, and wherein preferably said cells are human cells.

10. The method according to claim 1, wherein
said method is performed without a step of purifying and/or enriching said cells to be identified, preferably using whole blood and/or non-trypsinized tissue.

11. The method according to claim 1, further comprising the step of concluding on the immune and/or disease status of said mammal based on said immune cells as identified.

12. A method for monitoring the level of immune cells in a mammal,
comprising performing the method according to claim 6, and furthermore comparing said relative amount of said cells as identified to a sample taken earlier or in parallel from the same mammal, and/or to a control sample.

13. The method according to claim 1, further comprising measuring and/or monitoring the amount of said immune cells in response to chemical and/or biological substances that are provided to said mammal.

14. The method according to claim 1, wherein
said mammal suffers from or is likely to suffer from autoimmune diseases, transplant rejections, infection diseases, cancer, endometriosis, cardiovascular diseases, and/or allergy.

15. A kit for identifying, quantifying, and/or monitoring immune cells in a mammal based on the analysis of the bisulfite accessibility of CpG positions in the gene region according to SEQ ID No. 1, comprising components for performing a method according to claim 1, in particular a kit comprising a) a bisulfite reagent, and b) materials for the analysis of the methylation status of CpG positions selected from the CpG positions in the region according to SEQ ID NO: 1, such as an oligomer selected from the sequences according to SEQ ID NOs: 2 to 6.

16. An oligomer according to any of SEQ ID No. 2 to 6, the amplicon according to SEQ ID No. 1, or the bisulfite treated sequences thereof.

17. A method comprising using the kit of claim 15, for identifying, quantifying, and/or monitoring immune cells in a mammal.

18. A method comprising using the oligomer or amplicon according to claim 16 for identifying, quantifying, and/or monitoring immune cells in a mammal.
